(19) United States
(12) Patent Application Publication
(10) Pub. No.: US 2025/0262447 A1
(43) Pub. Date: Aug. 21, 2025

(54) PULSED ELECTROMAGNETIC FIELD THERAPY DELIVERY APPARATUS

(71) Applicant: Bradley CARDEN, Manly Vale (AU)

(72) Inventor: Bradley CARDEN, Manly Vale (AU)

(21) Appl. No.: 19/058,784

(22) Filed: Feb. 20, 2025

(30) Foreign Application Priority Data
Feb. 20, 2024 (AU) 2024900416

(51) Int. Cl. A61N 2/00; A61N 2/02

(52) U.S. Cl. CPC A61N 2/002; A61N 2/02

(57) ABSTRACT

A pulsed electromagnetic field (PEMF) therapy delivery apparatus is disclosed. The apparatus comprises a mat formed from an optically transparent material, a plurality of electromagnetic field generating coils distributed across the mat, and control circuitry electrically connected to the coils. The mat is configured for placement on a red light therapy light source, allowing red light to propagate through the mat while the coils generate pulsed electromagnetic fields to provide simultaneous PEMF and red light therapy. The control circuitry may regulate pulse waveform, frequency, and field strength and may be wirelessly controlled. Pressure sensors may selectively activate coils based on detected pressure. The mat may further include embedded LEDs and microvibrators, which may be selectively activated based on detected pressure or ambient light levels. The apparatus may be preconfigured with therapeutic programs defining operational parameters for targeted therapy applications.

### FIELD OF THE INVENTION

The invention relates to therapeutic devices, and more particularly to an apparatus configured to deliver pulsed electromagnetic field (PEMF) therapy. The apparatus is designed for integration with red light therapy systems, enabling simultaneous application of PEMF and red light therapy.

### BACKGROUND OF THE INVENTION

Pulsed electromagnetic field (PEMF) therapy and red light therapy are widely used therapeutic modalities, each providing distinct physiological benefits. PEMF therapy applies electromagnetic fields to stimulate cellular function, enhance circulation, and promote tissue regeneration. It has been used in various medical and wellness applications, including pain management, muscle recovery, and bone healing. By inducing small electrical currents in biological tissues, PEMF therapy influences cellular activity, supporting ATP production, reducing inflammation, and improving neuromuscular function.

Red light therapy, including both visible red and near-infrared (NIR) wavelengths, is known for its ability to penetrate the skin and underlying tissues to stimulate cellular repair and regeneration. It is frequently applied in dermatology, physiotherapy, and rehabilitation settings to promote collagen production, reduce inflammation, and enhance blood flow. Red light in the 600-1200 nm range has been observed to interact with mitochondria, leading to improved cellular energy production and metabolic activity.

### SUMMARY OF THE DISCLOSURE

A pulsed electromagnetic field (PEMF) therapy delivery apparatus is disclosed. The apparatus comprises a mat, a plurality of electromagnetic field generating coils distributed across the mat, and control circuitry electrically connected to the coils to generate pulsed electromagnetic fields. The mat is formed from an optically transparent material and is configured for placement on an existing red light therapy light source, allowing red light emitted from the light source to propagate through the mat while the coils generate pulsed electromagnetic fields to provide simultaneous PEMF therapy and red light therapy. The apparatus is designed to be retrofitted onto commercially available red light therapy devices, such as therapy beds and panels, without obstructing or significantly attenuating light transmission. This enables seamless integration with existing therapy systems, eliminating the need for additional standalone PEMF equipment while maintaining full red light exposure.

The selection of materials and coil placement minimises interference with red light propagation, allowing efficient energy transfer while preserving the intended therapeutic effects of red light therapy. By employing optically transparent conductive materials and strategic coil positioning, the apparatus reduces light occlusion, ensuring that red light effectively reaches the user's body while PEMF therapy is applied. The ability to incorporate PEMF functionality into existing red light therapy devices enhances usability in clinical and wellness settings, reducing equipment redundancy and optimising space efficiency.

In addition to reducing overall treatment time by combining two therapies into a single session, the apparatus provides enhanced therapeutic synergy through the concurrent application of electromagnetic and photobiomodulation effects. The targeted pulsed electromagnetic fields stimulate cellular activity, improving ion exchange and ATP production, while red light therapy promotes mitochondrial function and tissue repair. The integration of these mechanisms may result in more effective treatment outcomes compared to sequential therapy applications, particularly in musculoskeletal recovery, pain management, and inflammation reduction.

The control circuitry may be configured to generate pulsed electromagnetic fields with selectable pulse waveforms, including sine, triangle, sawtooth, rectangle, and square waveforms. A carrier frequency of 27.12 MHz may be applied to the coils in combination with a pulse repetition frequency in the range of 0.01 Hz to 10,000 Hz. The electromagnetic field strength may be adjustable within a range of 0.1 mT to 2 T.

The coils may be arranged in a central region of the mat, with power delivery wiring positioned along the mat's edge. The mat may be formed from flexible materials, allowing for adaptation to different therapy environments. The coils may be located between top and bottom layers of the mat, and at least one of these layers may include machined recess formations to accommodate the control circuitry and coils.

In embodiments, the mat is designed to be rolled up or folded along a fold line, with the coils positioned to avoid the fold line. The coils may be formed using metallic wires, vapour deposition, or chemical printing, and may include a conductive transparent material such as indium tin oxide to maintain optical transparency.

A controller may operably control the control circuitry and may be configured to regulate operation, duration, pulse waveform, pulse frequency, and field strength. The controller may receive control signals wirelessly from an electronic communication device. The control circuitry may include pressure sensors configured to selectively energise the coils based on detected pressure. Individual pressure sensors may allow for selective activation of specific coils.

The mat may incorporate a plurality of LEDs configured to emit red light, with wavelengths between 600 nm and 1200 nm for standalone application in the absence of external light source apparatus. A light sensor may be used to selectively activate the LEDs when external red light levels fall below a predetermined threshold. The mat may also include microvibrators for generating therapeutic vibrations, with the microvibrators positioned concentrically with the coils.

The controller may be preconfigured with a plurality of therapeutic programs stored in a memory device, with each program defining operational parameters such as pulse waveform, pulse frequency, field strength, LED activation, and microvibrator operation. The controller may adjust the operation of the coils, LEDs, or microvibrators based on the selected therapy program. Updates may be received via a data interface to modify or add new therapeutic programs.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a perspective view of a pulsed electromagnetic field (PEMF) therapy delivery apparatus.

FIG. 2 shows a disassembled perspective view of the apparatus, illustrating the top and bottom layers of the mat and the placement of the control circuitry and electromagnetic field generating coils therebetween.

FIG. 3 shows a red light therapy light source in the form of a light bed having an openable lid and a base with lamps therein, wherein the apparatus is placed on the base to simultaneously provide PEMF therapy and red light therapy.

FIG. 4 shows a top plan view of the apparatus, illustrating the arrangement of the electromagnetic field generating coils and control circuitry.

FIG. 5 shows the control circuitry and coils, with an optional controller for adjusting therapy parameters.

FIG. 6 shows a perspective view of a coil with an associated printed circuit board (PCB) located adjacent to the coil.

FIG. 7 shows a control schematic for the apparatus, including components for wireless communication, power delivery, and therapy parameter adjustments.

FIG. 8 shows a layer of the mat with machined circular recesses and a coil being inserted into a recess, wherein the recess closely conforms to the geometry of the coil.

### DESCRIPTION OF EMBODIMENTS

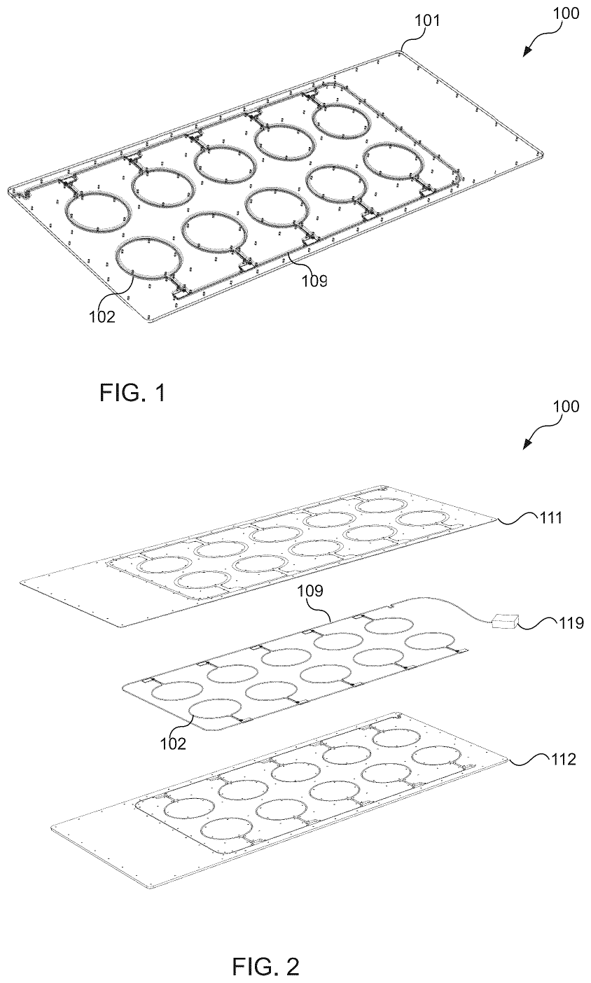

Pulsed electromagnetic field (PEMF) therapy delivery apparatus 100 is shown in FIG. 1 and comprises a mat 101 configured to enable the simultaneous application of PEMF therapy and red light therapy. The mat 101 is constructed from an optically transparent material, such as silicone, polyvinyl chloride (PVC), or acrylic, allowing red light to propagate through the mat while housing the electromagnetic field generating components.

As shown in FIG. 2, the mat 101 incorporates a plurality of electromagnetic field generating coils 102 distributed across its surface. These coils 102 are arranged to provide uniform electromagnetic field coverage and are electrically connected to control circuitry 103, which is configured to generate pulsed electromagnetic fields. The control circuitry 103 regulates the operation of the coils 102 in accordance with predefined operating parameters to ensure therapeutic PEMF treatment.

The transparent material of the mat 101 may be specifically selected to allow the transmission of red light in the wavelength range of 600 nm to 1200 nm, ensuring optimal therapeutic exposure to both visible red light and near-infrared (NIR) light. Materials such as optically clear silicone, flexible polyvinyl chloride (PVC), and cast or extruded acrylic may be used to provide high transmission efficiency while maintaining structural integrity. Silicone may be preferred for its flexibility and durability, allowing the mat 101 to conform to different surfaces while maintaining optical clarity. PVC offers a balance between transparency and mechanical strength, making it suitable for applications requiring a more rigid or semi-flexible structure. Acrylic, known for its high optical transmittance, may be advantageous in embodiments requiring maximum light penetration while providing resistance to wear and impact. The material selection ensures minimal absorption and scattering of light in the therapeutic range, allowing effective red light therapy while simultaneously accommodating the embedded electromagnetic field generating coils 102 and control circuitry 103.

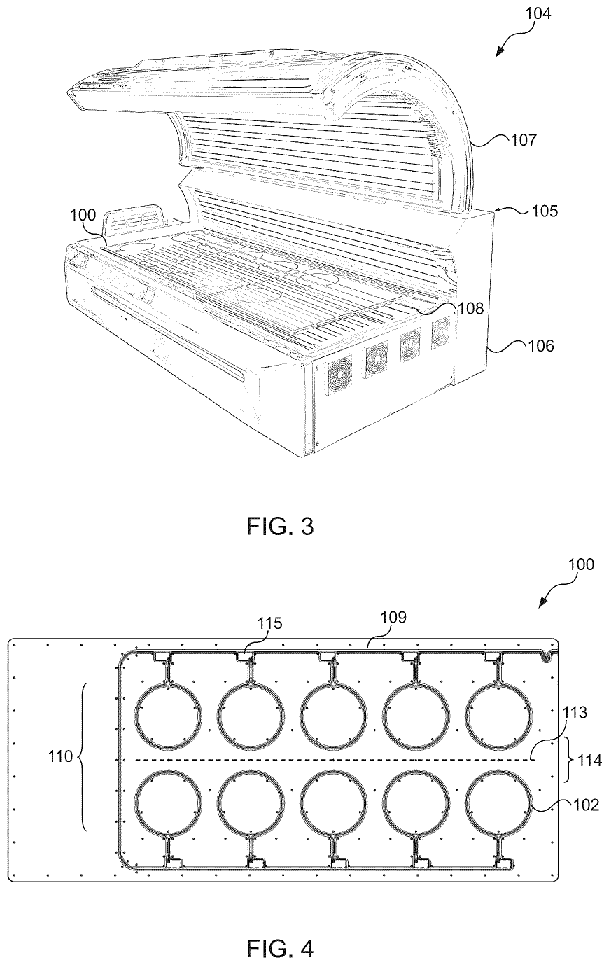

FIG. 3 illustrates the integration of the apparatus 100 with a red light therapy light source 104. In this embodiment, the red light therapy light source 104 comprises a conventional light bed 105, which includes a light bed base 106 and lid 107 which house a plurality of lamps 108 configured to emit red light. The apparatus 100 is placed on the base 106 of the light bed 105, allowing the red light emitted by the lamps 108 to propagate through the optically transparent mat 101. This configuration enables the simultaneous application of PEMF therapy and red light therapy.

The control circuitry 103 is designed to selectively activate the electromagnetic field generating coils 102 to generate pulsed electromagnetic fields while red light therapy is applied. When the apparatus 100 is in operation, the control circuitry 103 supplies an alternating or pulsed direct current to each coil 102 according to a predefined pulse waveform, frequency, and intensity. As current flows through the coil 102, a time-varying magnetic field is generated in accordance with Faraday's Law of Induction. This magnetic field extends outward from the coil 102 and interacts with biological tissues placed in proximity to the mat 101. The generated electromagnetic field induces small electric currents, known as eddy currents, in the conductive biological tissues according to Lenz's Law. These induced currents influence ion movement across cell membranes, modulating cellular function and promoting processes such as increased ATP production, improved microcirculation, and enhanced tissue regeneration.

The combined effect of PEMF therapy and red light therapy enhances therapeutic outcomes by stimulating cellular activity through electromagnetic fields while simultaneously promoting tissue recovery through red light exposure.

The control circuitry 103 may be configured to generate pulsed electromagnetic fields by energising the electromagnetic field generating coils 102 with waveforms selected from sine, triangle, sawtooth, rectangle, and square waveforms. These waveform options allow for variation in the electromagnetic pulses applied to the user, enabling different therapeutic effects based on the desired treatment parameters. In a preferred embodiment, the user may select or adjust the waveform type depending on the intended application.

In some embodiments, the control circuitry 103 may apply a carrier frequency of 27.12 MHz to the coils 102 in combination with a pulse repetition frequency in the range of 0.01 Hz to 10,000 Hz. The carrier frequency of 27.12 MHz may be advantageous as it falls within the industrial, scientific, and medical (ISM) radio band, which may help minimise interference with other electronic devices while optimising the depth of electromagnetic field penetration into biological tissues. The pulse repetition frequency may be adjustable within the defined range to modulate the therapeutic effect, allowing the system to accommodate different treatment protocols.

The electromagnetic field strength generated by the coils 102 may be adjustable within a range of 0.1 mT to 2 T. This range may allow for both low-intensity and high-intensity PEMF therapy, accommodating various therapeutic applications. Lower field strengths may be used for general wellness and relaxation, whereas higher field strengths may be beneficial for applications such as bone regeneration or deep tissue stimulation. In some embodiments, the control circuitry 103 may allow for real-time adjustment of the field strength based on user input or pre-programmed therapy protocols.

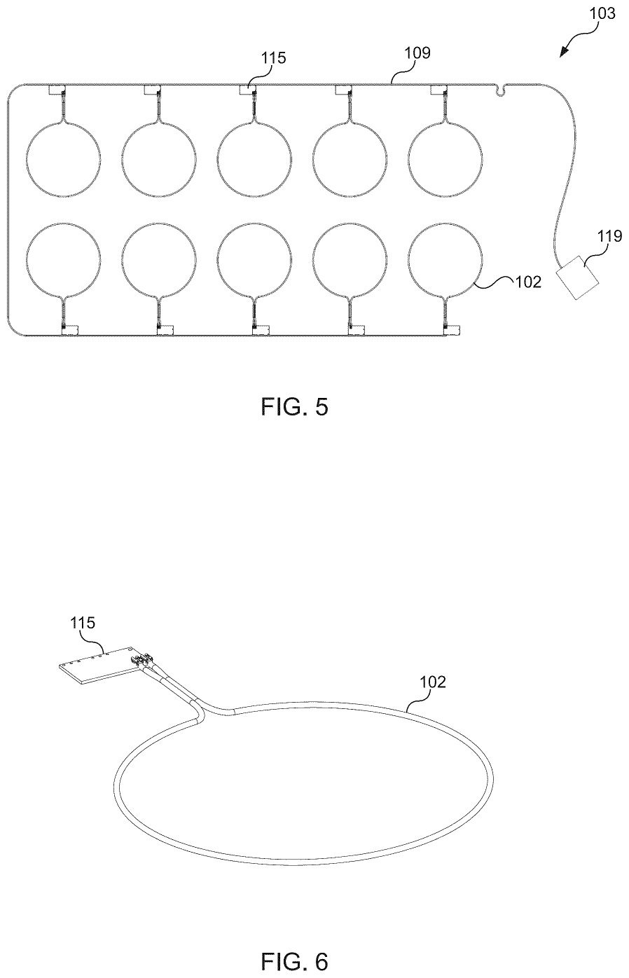

In a preferred embodiment shown in FIG. 4, the electromagnetic field generating coils 102 are positioned in a central region 110 of the mat 101 to optimise electromagnetic field coverage while non-electromagnetic field componentry is routed around the edges of the mat 101 minimising interference with light propagation. This arrangement may enhance the uniformity of therapy while ensuring that light transmission remains effective.

The mat 101 may be rectangular, with the coils 102 centrally arranged along its length to provide an efficient distribution of electromagnetic energy. This configuration may help align the PEMF therapy with key treatment areas of a user's body while ensuring consistent exposure to red light. By maintaining a structured layout, the apparatus 100 may be compatible with various red light therapy beds 105 while maintaining a balanced ratio between electromagnetic stimulation and light transmission.

To further reduce obstruction of red light transmission, power delivery wiring 109 may be routed along an edge of the mat 101, rather than through the light-transmitting central region 110. In some embodiments, the wiring 109 may be routed perpendicularly from the edge of the mat 101 toward each coil 102, ensuring minimal interference with light propagation. This arrangement may also facilitate a compact and low-profile design, preventing unnecessary shadowing or blocking of the red light.

The electromagnetic field generating coils 102 may be circular to enhance the uniformity and strength of the generated electromagnetic field. The circular geometry may be preferred as it allows for a more symmetrical and efficient magnetic flux distribution, reducing edge effects and optimising the therapeutic impact. Furthermore, the circular design may minimise unnecessary material usage, improving transparency in regions surrounding each coil.

In some embodiments, the coils 102 may have a diameter in excess of 150 mm to provide broader electromagnetic coverage while maintaining sufficient spacing to allow red light to propagate effectively through the mat 101. In certain implementations, the diameter may be further increased to exceed 200 mm, optimising field strength while continuing to preserve light permeability. The selection of coil size may depend on specific therapeutic requirements and the dimensions of the red light therapy bed 105 in which the apparatus 100 is used.

The mat 101 may be flexible, allowing it to conform to different surfaces and body positions during use. Flexibility may enhance user comfort and facilitate compatibility with various therapy setups, including integration with red light therapy beds 105 or placement on other support surfaces. In some embodiments, the flexibility of the mat 101 may enable it to be rolled or folded for compact storage and transport, making it suitable for both clinical and personal use.

In a preferred embodiment, the mat 101 may be formed from silicone, which provides both durability and optical transparency for efficient light transmission. Silicone may also offer resistance to heat, moisture, and wear, making it well-suited for repeated use in therapy environments. Additionally, silicone may provide a soft and pliable surface that improves comfort during treatment sessions.

As shown in FIG. 2, the mat 101 may comprise a top layer 111 and a bottom layer 112, with the control circuitry 103 and electromagnetic field generating coils 102 located therebetween. This layered construction may protect the internal components from mechanical stress, moisture, and external contaminants while maintaining a smooth, transparent outer surface. The placement of the control circuitry 103 and coils 102 between the layers may also contribute to a uniform thickness and structural integrity of the mat 101, preventing surface irregularities that could affect user comfort or light transmission.

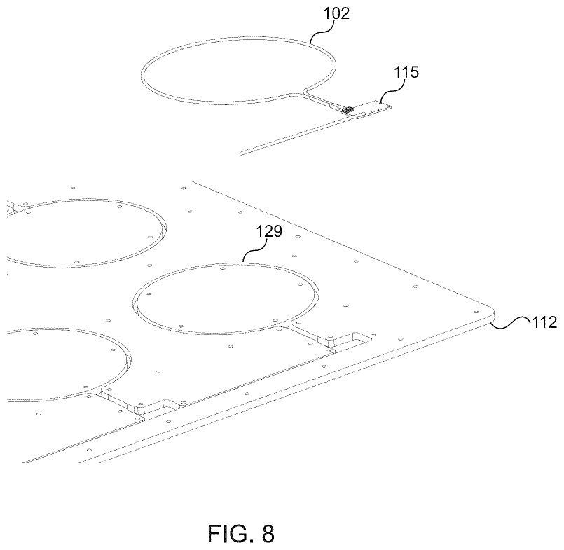

The mat 101 may comprise a top layer 111 and a bottom layer 112, wherein at least one of these layers includes machined recess formations 129 to accommodate the control circuitry 103 and electromagnetic field generating coils 102. As shown in FIG. 8, the recess formations 129 may be precisely machined to closely conform to the geometry of the coils 102, ensuring secure placement while maintaining a low-profile structure. This recessed configuration may help minimise the overall thickness of the mat 101, reducing bulk and enhancing flexibility while ensuring the coils 102 remain in a stable position during use.

In some embodiments, at least one of the layers 111, 112 may be formed from acrylic, which may provide structural rigidity while maintaining optical transparency to facilitate red light transmission. Acrylic may also offer high durability and resistance to wear, making it suitable for repeated use in therapy environments. The combination of a flexible base material, such as silicone, with a rigid or semi-rigid acrylic layer may provide an optimal balance between flexibility, structural integrity, and light transmission efficiency.

The mat 101 may be configured to be rolled up, allowing for convenient storage and transport. In some embodiments, the flexibility of the mat 101, combined with the selection of materials such as silicone or acrylic, may enable it to be rolled without damaging the internal components. Rolling the mat 101 may be particularly advantageous for portable or home-use applications, where compact storage is beneficial.

In a preferred embodiment, the mat 101 may be configured to be folded in half along a fold line 113 as is shown in FIG. 4, providing an alternative compact storage method while maintaining structural integrity. The fold line 113 may be defined by a region of reduced material thickness or a flexible hinge section, allowing the mat 101 to be folded without placing excessive stress on internal components.

To preserve functionality and prevent damage, the electromagnetic field generating coils 102 may be positioned away from a coil-free fold region 114 to avoid the fold line 113. This arrangement may reduce the risk of wire breakage or deformation of the coils 102 during folding. Additionally, the power delivery wiring 109 may be routed around the fold line 113 to further minimise strain on electrical connections. By strategically positioning the internal components, the mat 101 may maintain both durability and performance even when subjected to repeated folding or rolling.

In one embodiment, the electromagnetic field generating coils 102 may be formed from metallic wires embedded within the mat 101. Metallic wire coils may provide a robust and efficient means of generating pulsed electromagnetic fields while maintaining a degree of flexibility. The use of fine-gauge wire may allow for a balance between conductivity and minimal obstruction to light transmission, ensuring that red light from the underlying red light therapy light source 104 can effectively propagate through the mat 101.

In an alternative embodiment, the coils 102 may be formed by vapour deposition, wherein a thin conductive layer is deposited onto a substrate within the mat 101 to create a coil pattern. This method may enable the formation of extremely fine and uniform conductive traces, reducing material thickness and increasing light transmission through the mat 101. Vapour-deposited coils may also enhance flexibility by eliminating the need for bulky wiring, making the mat 101 more adaptable to curved surfaces or foldable configurations.

In another embodiment, the coils 102 may be formed by chemical printing, wherein conductive inks or solutions are applied to a flexible substrate to create the coil structure. Chemical printing techniques, such as inkjet or screen printing, may allow for precise control over the coil geometry while maintaining a low profile. Printed coils may also be embedded seamlessly between the top layer 111 and bottom layer 112 of the mat 101, preserving transparency and mechanical flexibility.

In some embodiments, the coils 102 may comprise a conductive transparent material to further enhance light transmission through the mat 101. This may allow for an improved balance between electromagnetic field generation and red light propagation. In a preferred embodiment, the conductive transparent material may be indium tin oxide (ITO), which exhibits both electrical conductivity and high optical transparency. The use of ITO may enable the formation of coils that generate pulsed electromagnetic fields while minimising interference with the passage of red light, optimising the dual therapy effect of the apparatus 100.

The control circuitry 103 may comprise a printed circuit board (PCB) 115 located adjacent to each electromagnetic field generating coil 102, as shown in FIG. 6. Each PCB 115 may include an oscillator circuit 125 configured to generate pulsed electromagnetic fields by energising the respective coil 102. The oscillator circuit 125 may be designed to operate at a carrier frequency of 27.12 MHz, with a pulse repetition frequency in the range of 0.01 Hz to 10,000 Hz, allowing for precise modulation of the electromagnetic field characteristics.

In some embodiments, a direct current (DC) power supply may be provided to each PCB 115, enabling independent operation of each coil 102. This distributed arrangement may allow for greater control over the spatial distribution of electromagnetic fields, as each PCB 115 can be individually regulated to adjust field strength, pulse waveform, and frequency parameters. The use of localised PCBs 115 rather than a single centralised control unit may also reduce power losses associated with long conductive traces and may improve system efficiency.

By positioning the PCBs 115 adjacent to the coils 102, this configuration may minimise electromagnetic interference between components, as the close proximity of the oscillator circuit 125 to each coil 102 may reduce unintended signal degradation or crosstalk between coils. Additionally, the distributed nature of the control circuitry 103 may enhance redundancy, as a failure in one PCB 115 does not compromise the functionality of the entire apparatus 100.

Furthermore, this arrangement may contribute to overall flexibility, as the modular design allows for the selective activation of coils 102 in different regions of the mat 101. This capability may enable targeted PEMF therapy based on specific treatment areas while maintaining the simultaneous application of red light therapy.

The apparatus 100 may further comprise a controller 119 operably controlling the control circuitry 103. The controller 119 may be configured to regulate at least one of operation, operational duration, pulse waveform, pulse frequency, and field strength. By dynamically adjusting these parameters, the controller 119 may allow for personalised therapy settings tailored to specific user requirements. The controller 119 may execute pre-programmed treatment protocols or be configured for real-time manual adjustments, enabling flexibility in therapy application.

In some embodiments, the controller 119 may be configured to receive control signals wirelessly from an electronic communication device 126. The electronic communication device 126 may take the form of a mobile phone, tablet, or dedicated remote-control device, with a software application installed thereon specifically designed to interface with the apparatus 100. The software application may enable users to control various modes of operation, select therapeutic programs, and adjust PEMF treatment parameters such as pulse frequency based on the desired treatment effect.

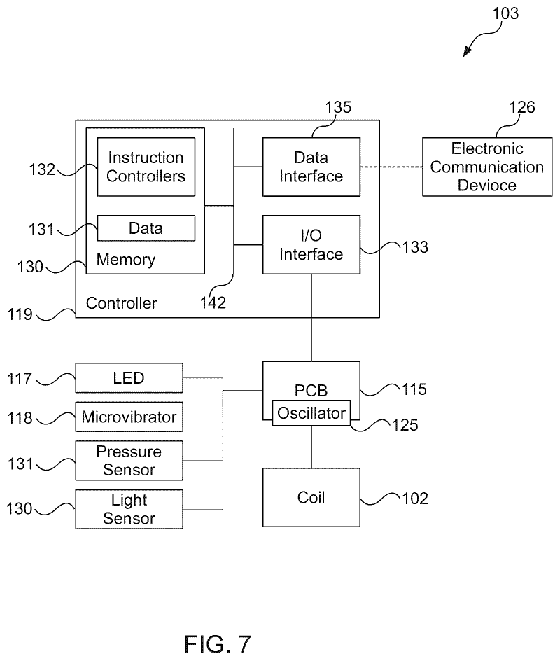

FIG. 7 shows an exemplary control circuitry 103 of the apparatus 100 comprising the controller 119. The controller 119 may comprise a processor configured for processing digital data and being in operable communication with a memory device 130 via a system bus 142. The memory device 130 is configured for storing digital data 131 and computer program code instructions which may be logically divided into a plurality of computer program code instruction controllers 132. In use, the processor fetches these instructions and associated data from the memory 130 for interpretation and execution of the functionality described herein. FIG. 7 further shows the processor being in operable communication with a data interface 135 for sending and receiving data across data networks, including local, wide area wireless and wired data networks, including for communication with associated electronic communication devices 134. FIG. 7 further shows the processor comprise an I/O interface 133 configured for interfacing with various peripheral componentry shown.

The electronic communication device 126 may be configured to transmit therapy application data across a data network, such as the Internet, to enable remote monitoring and analysis. The data interface 128 may facilitate the transmission of real-time therapy data, including session duration, waveform selection, and electromagnetic field strength, which may be logged for future reference. In some embodiments, the apparatus 100 may also be configured to receive control instructions from remote sources via the Internet, allowing healthcare providers or wellness professionals to adjust treatment settings remotely based on user needs.

A wireless data interface 127 may support various communication protocols, such as Bluetooth, Wi-Fi, or cellular connectivity, ensuring compatibility with different electronic communication devices 126 and enabling seamless integration into connected health platforms. The ability to transmit and receive data across a network may enhance the usability of the apparatus 100, providing users with remote access to treatment analytics, therapy customisation, and even automated adjustments based on historical treatment patterns.

The control circuitry 103 may comprise at least one pressure sensor 131 configured to detect applied pressure and selectively energise the electromagnetic field generating coils 102 in response. The pressure sensor 131 may be integrated within the mat 101, positioned between the top layer 111 and bottom layer 112, to detect user presence or contact pressure. In a preferred embodiment, the pressure sensor 131 may be implemented using capacitive, resistive, or piezoelectric sensing technologies, allowing for accurate and responsive pressure detection.

Capacitive pressure sensors may detect changes in capacitance caused by pressure exerted on the mat 101, providing a highly sensitive and low-power solution. Resistive pressure sensors may measure variations in electrical resistance due to mechanical deformation, offering a cost-effective approach. Piezoelectric pressure sensors may generate an electrical signal in response to applied pressure, providing rapid response times suitable for dynamic therapy adjustments.

In some embodiments, the control circuitry 103 may include individual pressure sensors 131 corresponding to each electromagnetic field generating coil 102. Each pressure sensor 131 may be positioned adjacent to or beneath a respective coil 102, allowing for precise detection of pressure distribution across the mat 101. The control circuitry 103 may be configured to selectively energise individual coils 102 based on detected pressure, ensuring that PEMF therapy is applied only to regions where user contact is present.

This individualised control may offer several advantages. By activating only the coils 102 in direct contact with the user, energy efficiency may be improved, reducing unnecessary power consumption and minimising heat generation. Additionally, selective activation of coils 102 may allow for targeted therapy, ensuring that PEMF stimulation is applied precisely to the areas requiring treatment. This feature may be particularly beneficial in therapeutic applications where localised treatment is desired, such as pain management or injury recovery.

The integration of pressure sensors 131 may also enhance user experience by enabling adaptive therapy modes. For example, the control circuitry 103 may adjust field strength dynamically based on detected pressure levels, providing a more intuitive and responsive therapy session. Furthermore, by detecting variations in user positioning, the apparatus 100 may adjust coil activation patterns in real-time to maintain consistent treatment efficacy.

The mat 101 may further comprise a plurality of LEDs 117 configured to emit red light, allowing the apparatus 100 to function as a standalone therapy device when an external red light therapy light source 104 is not available. The LEDs 117 may be embedded within the mat 101, positioned between the top layer 111 and bottom layer 112, and arranged in a pattern that ensures uniform illumination across the treatment surface.

In a preferred embodiment, the LEDs 117 may be configured to emit red light within a wavelength range of 600 nm to 1200 nm. This range encompasses both visible red light (typically around 600-700 nm) and near-infrared (NIR) light (typically around 700-1200 nm), both of which are known for their therapeutic benefits. Red light within the lower end of the spectrum may penetrate the skin to stimulate cellular activity and collagen production, while NIR wavelengths may penetrate deeper into muscle and tissue, promoting circulation and reducing inflammation. The ability to cover a broad spectrum of red and near-infrared light may provide enhanced therapeutic benefits.

In some embodiments, the apparatus 100 may include embedded light sensors 130 configured to detect the presence of external red light from a separate red light therapy light source 104. These light sensors 130 may be positioned on the surface or within the layers of the mat 101, oriented to measure ambient light levels. The control circuitry 103 may be configured to selectively activate the LEDs 117 only when an external red light source is not detected, thereby optimising energy efficiency and reducing unnecessary power consumption.

When the mat 101 is placed on an operational red light therapy bed 105, the light sensors 130 may detect sufficient external illumination, preventing unnecessary activation of the embedded LEDs 117. Conversely, if the apparatus 100 is used in an environment without an external red light source, the control circuitry 103 may automatically energise the LEDs 117 to provide the necessary red light therapy. This functionality may enable seamless operation in both integrated and standalone use cases, ensuring that users receive consistent therapy regardless of the availability of an external light source.

Furthermore, the control circuitry 103 may be configured to modulate the intensity of the LEDs 117 based on detected light levels. If an external light source provides only partial illumination, the embedded LEDs 117 may activate at a reduced intensity to supplement the available red light, maintaining optimal therapeutic exposure without excessive energy consumption.

The mat 101 may further comprise a plurality of microvibrators 118 configured to generate therapeutic vibrations, thereby enhancing the therapeutic efficacy of the apparatus 100. These microvibrators 118 may be embedded within the layers of the mat 101 and positioned to deliver mechanical stimulation to the user during therapy. The vibrations may be applied continuously or in pulsed patterns, either independently or in synchronization with the pulsed electromagnetic fields generated by the electromagnetic field generating coils 102.

In a preferred embodiment, the microvibrators 118 may be piezoelectric, wherein each microvibrator 118 comprises a piezoelectric element that generates mechanical vibrations in response to an applied electrical signal. Piezoelectric microvibrators may offer high precision, low power consumption, and compact size, allowing them to be integrated seamlessly within the mat 101. Alternative embodiments may employ electromagnetic or voice-coil-based microvibrators, depending on the desired vibration characteristics.

To optimize the combined therapeutic effect of PEMF therapy and mechanical stimulation, the microvibrators 118 may be concentrically located with respective coils 102, as shown in FIG. 6. By aligning each microvibrator 118 with a corresponding coil 102, the apparatus 100 may deliver simultaneous electromagnetic and mechanical stimulation to the same treatment region, enhancing the overall therapeutic outcome. This concentric arrangement may ensure that the vibrations are focused precisely where the PEMF field is strongest, maximizing neuromuscular stimulation and circulation enhancement. The integration of microvibrators 118 may stimulate blood flow and

lymphatic circulation, complementing the cellular effects of PEMF therapy. Vibrations may also promote relaxation by reducing muscle tension and may enhance pain relief through the activation of mechanoreceptors in the skin and deeper tissues. Additionally, the combination of PEMF therapy, red light therapy, and vibrational therapy in a single apparatus 100 may offer a more comprehensive treatment solution, reducing the need for multiple separate therapeutic devices.

In some embodiments, the control circuitry 103 may be configured to adjust the frequency and intensity of the vibrations based on user input or pre-programmed therapeutic settings. The microvibrators 118 may be operated in synchronization with the PEMF pulses, applying mechanical stimulation in tandem with electromagnetic field delivery to reinforce neuromuscular and tissue responses. The ability to modulate vibration parameters may allow for customization based on specific treatment goals, such as deep tissue stimulation, relaxation, or pain relief.

In some embodiments, the control circuitry 103 may be configured to selectively control the operation of the LEDs 117 and microvibrators 118 based on input from pressure sensors 131 embedded within the mat 101. These pressure sensors may be positioned between the top layer 111 and bottom layer 112 of the mat 101, allowing for detection of user presence and pressure distribution. By utilizing pressure data, the apparatus 100 may dynamically adjust therapy delivery, ensuring that red light emission and vibrational therapy are applied only to regions where user contact is detected.

In one embodiment, the control circuitry 103 may selectively energise the LEDs 117 depending on pressure detection. If a user is positioned on a specific area of the mat 101, the pressure sensors 131 may detect the applied force and signal the control circuitry 103 to activate the corresponding LEDs 117 in that region. This configuration may enhance energy efficiency by ensuring that red light therapy is only applied where it is needed, rather than uniformly across the entire mat 101. Additionally, this selective activation may help prevent unnecessary light exposure to non-contact areas, optimising treatment effectiveness.

In an alternative embodiment, the control circuitry 103 may comprise individual pressure sensors 131 associated with respective LEDs 117, enabling precise control over each light-emitting region. Each LED 117 may be selectively activated based on pressure detected in its corresponding area, allowing for a highly localized and adaptive red light therapy session. This individual control may be beneficial for targeting specific treatment zones, such as sore muscles, joints, or areas of localized pain.

Similarly, the control circuitry 103 may be configured to selectively activate the microvibrators 118 based on detected pressure. When pressure is applied to a particular section of the mat 101, the corresponding microvibrators 118 may be energised to provide mechanical stimulation to the contact area. This functionality may enhance the therapeutic effect by ensuring that vibration therapy is applied only where physical contact is detected, maximizing comfort and efficiency.

In a preferred embodiment, the control circuitry 103 may include individual pressure sensors 131 assigned to each microvibrator 118, allowing for independent activation of vibrational therapy zones. This configuration may be particularly advantageous in applications where targeted muscle relaxation or circulation enhancement is desired. The pressure-based activation may also enable an adaptive therapy mode, wherein the intensity of vibration is modulated based on the level of detected pressure, providing a more responsive and personalised treatment experience.

By integrating pressure sensors 131 to selectively control LEDs 117 and microvibrators 118, the apparatus 100 may offer an intelligent, energy-efficient, and user-responsive therapy system. The ability to activate therapeutic components only where needed may not only conserve power but also enhance the overall effectiveness of PEMF, red light, and vibrational therapy, providing a more refined and adaptable treatment experience.

In some embodiments, the apparatus 100 may be configured with a plurality of preprogrammed therapeutic programs stored in a memory device 121 and executed by the controller 119. These preprogrammed therapy modes allow users to select treatment protocols tailored to specific ailments, with variations in PEMF pulse frequency, red light wavelength, vibration intensity, and operational duration. The controller 119 retrieves stored treatment profiles from the memory device 121 and adjusts the operational parameters of the control circuitry 103 accordingly.

For example, in one embodiment, the apparatus 100 may include therapy programs specifically designed for pain relief, muscle recovery, joint health, inflammation reduction, and general wellness. These programs may be selectable via an electronic communication device 126, such as a mobile phone, through a dedicated software application. The software application may provide a user interface allowing for the selection of a desired therapy mode, after which the controller 119 configures the apparatus 100 to operate according to the predefined settings.

The pulse frequency of the electromagnetic field generating coils 102 may be varied depending on the ailment being treated. For chronic pain management, a lower pulse repetition frequency, such as 1-50 Hz, may be used to stimulate endorphin release and provide analgesic effects. For bone healing and regeneration, a pulse frequency of approximately 75 Hz may be selected to promote osteoblast activity and enhance mineralization. For muscle recovery and relaxation, a frequency in the range of 10-100 Hz may be used to enhance circulation and reduce muscle tension. Higher frequencies, such as 1000 Hz or more, may be used for deep tissue stimulation and nerve regeneration.

The wavelength of red light emitted by the LEDs 117 may also be adjusted based on the selected therapeutic program. For superficial skin treatments, such as wound healing and collagen production, wavelengths in the range of 600-700 nm may be used to penetrate the upper layers of the skin and stimulate cellular repair. For deeper tissue applications, such as muscle recovery and joint inflammation, near-infrared wavelengths in the range of 800-1200 nm may be selected to achieve greater tissue penetration and enhance mitochondrial activity.

The microvibrators 118 may be controlled according to the selected therapy mode, with variations in vibration frequency and intensity. For muscle relaxation and circulation enhancement, lower vibration frequencies, such as 20-50 Hz, may be used to stimulate blood flow and lymphatic drainage. For deep tissue stimulation, higher vibration frequencies, such as 100-200 Hz, may be applied to target muscle adhesions and promote tissue recovery. In some embodiments, the microvibrators 118 may be synchronised with the PEMF pulse frequency to create a combined effect, enhancing neuromuscular activation and promoting faster recovery.

The integration of preprogrammed therapeutic modes enables the apparatus 100 to provide targeted treatment based on specific conditions while allowing users to easily switch between therapy modes. In some embodiments, the memory device 121 may store user therapy data, allowing for personalised treatment plans that adapt based on previous usage patterns. The controller 119 may further be configured to receive software updates via a data interface 128, enabling new therapy programs to be added and optimised over time.

In an exemplary method of use, the apparatus 100 may be positioned on a red light therapy light source 104, such as a light bed 105, for simultaneous application of PEMF therapy and red light therapy. The mat 101 is placed on the light bed base 106, ensuring that the electromagnetic field generating coils 102 are aligned with the targeted treatment area. If the apparatus 100 is used as a standalone device, the LEDs 117 embedded within the mat 101 may be activated to provide red light therapy without the need for an external light source.

Before initiating therapy, the user may select a desired treatment program through an electronic communication device 126, such as a mobile phone, tablet, or dedicated controller. The software application installed on the electronic communication device 126 may provide options to customise parameters such as pulse waveform, pulse frequency, field strength, red light wavelength, and vibration intensity. The selected treatment settings are transmitted wirelessly to the controller 119, which configures the control circuitry 103 to generate the appropriate PEMF signals and regulate the operation of the LEDs 117 and microvibrators 118.

Once the treatment program is initiated, the control circuitry 103 energises the electromagnetic field generating coils 102 according to the predefined settings. The oscillator circuit 125 on each PCB 115 generates a pulsed electromagnetic field, which is transmitted through the mat 101 and applied to the user's body. Simultaneously, red light emitted from either the red light therapy light source 104 or the LEDs 117 propagates through the optically transparent mat 101, providing concurrent light therapy. If the selected treatment mode includes mechanical stimulation, the microvibrators 118 are activated, applying targeted vibrations to the treatment area.

During use, the apparatus 100 may dynamically adjust therapy parameters based on real-time sensor feedback. If pressure sensors 131 detect user contact, the control circuitry 103 may selectively energise the coils 102, LEDs 117, or microvibrators 118 only in the regions where pressure is detected. In embodiments where individual pressure sensors 131 are integrated, each coil 102, LED 117, and microvibrator 118 may be controlled independently, allowing for precise, localised therapy application.

The therapy session may continue for a predetermined duration, as specified in the selected treatment program. The controller 119 may monitor elapsed time and automatically deactivate the apparatus 100 upon completion of the session. If the user wishes to extend or modify the treatment, they may adjust settings via the electronic communication device 126 or manually through an interface on the mat 101.

At the conclusion of the session, the apparatus 100 may be removed from the light bed 105 or stored in a compact form. In embodiments where the mat 101 is flexible, it may be rolled up or folded along a fold line 113 for easy transport and storage. The modular design of the apparatus 100 may allow it to be integrated with various therapy environments, including clinical, wellness, and home settings.

The exemplary method of use highlights the adaptability of the apparatus 100, allowing for both integrated and standalone operation. By combining PEMF therapy, red light therapy, and mechanical stimulation in a single system, the apparatus 100 provides an efficient, multi-modal therapeutic solution.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1. A pulsed electromagnetic field (PEMF) therapy delivery apparatus comprising a mat, a plurality of electromagnetic field generating coils distributed across the mat, and control circuitry electrically connected to the electromagnetic field generating coils to generate pulsed electromagnetic fields, wherein the mat is formed from an optically transparent material and configured for placement on a red light therapy light source in use such that red light emitted by the light source propagates through the mat while the electromagnetic field generating coils generate the pulsed electromagnetic fields to provide simultaneous PEMF therapy and red light therapy.

2. The apparatus of claim 1, wherein the control circuitry is configured to generate pulsed electromagnetic fields using at least one of a sine, triangle, sawtooth, rectangle, or square waveform.

3. The apparatus of claim 1, wherein the control circuitry is configured to apply a carrier frequency of 27.12 MHz to the electromagnetic field generating coils in combination with a pulse repetition frequency in a range of 0.01 Hz to 10,000 Hz.

4. The apparatus of claim 1, wherein the electromagnetic field generating coils are configured to generate a field strength in a range of 0.1 mT to 2 T.

5. The apparatus of claim 1, wherein the electromagnetic field generating coils are located in a central region of the mat and wherein power delivery wiring is located along an edge of the mat.

6. The apparatus of claim 5, wherein the mat is rectangular and wherein the electromagnetic field generating coils are centrally arranged along the mat.

7. The apparatus of claim 6, wherein the power delivery wiring is routed perpendicularly from the edge of the mat to each electromagnetic field generating coil.

8. The apparatus of claim 1, wherein each electromagnetic field generating coil is circular.

9. The apparatus of claim 8, wherein each electromagnetic field generating coil has a diameter in excess of 150 mm.

10. The apparatus of claim 9, wherein each electromagnetic field generating coil has a diameter in excess of 200 mm.

11. The apparatus of claim 1, wherein the mat is flexible.

12. The apparatus of claim 11, wherein the mat comprises silicone.

13. The apparatus of claim 1, wherein the mat comprises top and bottom layers with the control circuitry and the electromagnetic field generating coils located therebetween.

14. The apparatus of claim 13, wherein at least one of the top or bottom layers has machined recess formations to accommodate the control circuitry and the electromagnetic field generating coils.

15. The apparatus of claim 14, wherein at least one of the top or bottom layers comprises acrylic.

16. The apparatus of claim 1, wherein the mat is configured to be rolled up.

17. The apparatus of claim 1, wherein the mat is configured to be folded along a fold line.

18. The apparatus of claim 17, wherein the electromagnetic field generating coils are positioned to avoid the fold line.

19. The apparatus of claim 1, wherein the electromagnetic field generating coils are formed by vapour deposition.

20. The apparatus of claim 1, wherein the electromagnetic field generating coils are formed by chemical printing.

21. The apparatus of claim 1, wherein the electromagnetic field generating coils comprise a conductive transparent material.

22. The apparatus of claim 21, wherein the conductive transparent material is indium tin oxide.

23. The apparatus of claim 1, wherein the control circuitry comprises a printed circuit board located adjacent to each electromagnetic field generating coil and wherein each printed circuit board comprises an oscillator circuit configured to generate pulsed electromagnetic fields.

24. The apparatus of claim 1, further comprising a controller operably connected to the control circuitry, wherein the controller is configured to control at least one of operation, operational duration, pulse waveform, pulse frequency, and field strength.

25. The apparatus of claim 24, wherein the controller is configured to receive control signals wirelessly from an electronic communication device.

26. The apparatus of claim 1, wherein the control circuitry comprises a pressure sensor and wherein the control circuitry is configured to selectively energise the electromagnetic field generating coils depending on detecting pressure.

27. The apparatus of claim 26, wherein the control circuitry comprises individual pressure sensors, and wherein the control circuitry is configured to selectively energise respective electromagnetic field generating coils individually based on pressure detected by the individual pressure sensors.

28. The apparatus of claim 1, further comprising a plurality of LEDs configured to emit red light.

29. The apparatus of claim 28, wherein the LEDs are configured to emit red light in a wavelength between 600 nm and 1200 nm.

30. The apparatus of claim 28, further comprising a light sensor, wherein the control circuitry is configured to selectively energise the LEDs in response to detecting incident red light below a predetermined threshold.

31. The apparatus of claim 1, further comprising a plurality of microvibrators configured to generate therapeutic vibrations.

32. The apparatus of claim 31, wherein the microvibrators are piezoelectric.

33. The apparatus of claim 32, wherein the microvibrators are concentrically located with respective electromagnetic field generating coils.

34. The apparatus of claim 28, wherein the control circuitry comprises a pressure sensor, and wherein the control circuitry is configured to selectively energise the LEDs depending on detecting pressure.

35. The apparatus of claim 31, wherein the control circuitry comprises a pressure sensor, and wherein the control circuitry is configured to selectively energise the microvibrators depending on detecting pressure.

36. The apparatus of claim 1, wherein the controller is preconfigured with a plurality of therapeutic programs stored in a memory device and wherein each therapeutic program defines at least one of pulse waveform, pulse frequency, field strength, LED activation, or microvibrator activation.

37. The apparatus of claim 36, wherein the controller is configured to receive a user selection of a therapeutic program based on a targeted ailment and control the electromagnetic field generating coils, LEDs, or microvibrators accordingly.

38. The apparatus of claim 37, wherein the controller is configured to modify at least one of the pulse waveform, pulse frequency, LED wavelength, or vibration intensity based on the selected therapeutic program.

39. The apparatus of claim 1, wherein the controller is configured to receive updates via a data interface to modify or add therapeutic programs.

40. A method of providing pulsed electromagnetic field (PEMF) therapy and red light therapy, the method comprising placing a mat on a red light therapy light source, the mat comprising a plurality of electromagnetic field generating coils and control circuitry electrically connected to the coils, wherein the mat is optically transparent to allow red light emitted from the light source to propagate therethrough, and activating the control circuitry to generate pulsed electromagnetic fields while red light therapy is simultaneously applied.

41. The method of claim 40, further comprising selecting a therapeutic program from a plurality of preconfigured programs stored in a memory device, wherein the selected therapeutic program defines at least one of pulse waveform, pulse frequency and field strength, and operating the control circuitry to apply the selected parameters.

42. The method of claim 40, further comprising detecting pressure applied to the mat using a pressure sensor and selectively energising at least one of the electromagnetic field generating coils based on the detected pressure.

43. The method of claim 40, further comprising detecting ambient red light using a light sensor and selectively activating LEDs embedded in the mat when the detected ambient red light falls below a predetermined threshold.

44. The method of claim 40, further comprising wirelessly transmitting a control signal from an electronic communication device to the controller, wherein the control signal defines at least one operational parameter of the electromagnetic field generating coils and adjusting the operation of the electromagnetic field generating coils in response to the control signal.
